(19) United States
(12) Patent Application Publication
(10) Pub. No.: US 2024/0082527 A1
(43) Pub. Date: Mar. 14, 2024

(54) GAS FLOW DELIVERY SYSTEM WITH MAGNETIC COUPLING ASSEMBLY AND MAGNETIC COUPLING ASSEMBLY THEREFOR

(71) Applicant: RainMaker Solutions, Inc., El Segundo, CA (US)

(72) Inventor: Robert Gregory Stahl, Culver City, CA (US)

(21) Appl. No.: 17/942,175

(22) Filed: Sep. 12, 2022

(51) Int. Cl. A61M 16/08; A61M 16/00; A61M 16/06; F16L 37/00

(52) U.S. Cl. CPC A61M 16/0816; A61M 16/0066; A61M 16/06; F16L 37/004

(57) ABSTRACT

A gas flow delivery system for delivering a gas into headgear adapted to be worn by a user, is disclosed. The gas delivery system comprises a gas source, headgear having a user interface configured to receive at least the nose and/or mouth of a user within an internal region thereof, and a fluid circuit in communication with the gas source at one end and the internal region of the user interface of the headgear at another end. A magnetic coupling assembly is interposed in the fluid circuit. The magnetic coupling assembly includes a first magnetic coupling member and a second magnetic coupling member, where the first and second magnetic coupling members have opposing mating surfaces and define an axis along which the first and second magnetic coupling members magnetically engage one another. Each of the first and second magnetic coupling members comprise a main body portion and a retaining cap connected to the main body portion, the retaining cap and main body portion of each coupling member define an annular chamber in which a ring-shaped magnet is disposed. The retaining caps of each of the first and second magnetic coupling members also define the opposing mating surfaces of the first and second coupling members. At least a portion of the opposing mating surfaces may be transverse to the axis. The headgear comprises an interface input tube and the first magnetic coupling member is connected to the interface input tube. The second magnetic coupling member is connected to a gas delivery tube that defines at least part of the fluid circuit. The second magnetic coupling member may be separated from the first magnetic coupling member through the application of an axial pull force. The second magnetic coupling member may also be separated from the first magnetic coupling member through a torque created by the application of a force orthogonal to the axis to the second magnetic coupling member.

### TECHNICAL FIELD

The present disclosure relates to a gas flow delivery devices and systems used to deliver a flow of gas to an airway of a subject, and a magnetic coupling assembly for use in such gas glow delivery systems.

### BACKGROUND

Gas flow delivery systems are used to deliver a flow of gas to an airway of a subject. Such systems have been used in the medical field to deliver gas to a patient. Examples of gas flow delivery systems in the medical field include a ventilator or respirator, which replaces or supplements a patient's respiration, and a pressure support system, which provides a flow of gas to an airway of a patient at an elevated pressure to treat a medical disorder, such as obstructive sleep apnea (OSA). Pressure support systems include, but are not limited to, continuous positive airway pressure (CPAP) devices, which deliver a constant positive pressure of air to the airway of a patient over multiple respiratory cycles, and variable pressure devices, where the pressure of the flow of gas delivered to the patient is variable.

Variable pressure support devices include auto-titrating devices that are capable of changing a base pressure or pressure profile delivered to the patent based on a monitored condition of the patient. Other variable pressure devices change the pressure of the flow of gas during a respiratory cycle. For purposes of the present patent disclosure, all systems that deliver a flow of gas or pressurized gas to an airway of a subject are referred to as gas flow delivery systems.

A typical gas flow delivery system comprises a pressure/flow generating system that produces a flow of gas for delivery to a subject and a system for communicating the flow of gas to the subject. The latter system typically includes a flexible conduit having one end coupled to a pressure/flow generating device or gas source and a second end portion that couples to an airway of the subject by means of a headgear interface assembly worn by the user. The conduit, which is also referred to as an air hose or patient circuit, carries the flow of gas from the pressure generating device during operation of the system. The headgear interface assembly includes a user interface device, typically in the form of a nasal, oral, or nasal/oral mask, and a headgear retaining assembly that attaches to the interface device so as to position the interface device over nose, mouth or nose and mouth of the user. The second end portion of the conduit is coupled to the user interface device to communicate the flow of gas to the airway of the user.

The components of the gas flow delivery system are coupled together such that they can be assembled and disassembled by a user or caregiver. For example, the user interface device (e.g., mask) may be selectively coupled to the distal end of the patient circuit, and the pressure/flow generating device may be selectively coupled to the proximal end of the patient circuit.

In a conventional gas flow delivery system, mechanical forms of connections have been used to join these various members to one another. For example, friction fittings are typically used to join the ends of the air hose to the headgear interface assembly and the pressure/flow generating device. While these techniques are suitable for the purpose of joining two components of the gas flow delivery system to one another, they can be difficult to assemble and disassemble for users with impaired physical and/or mental ability.

One attempt to solve this problem is described in U.S. Patent Publication No. 2010/0307497 to Busch. Busch discloses a magnetic coupling that is interposed in the gas flow path between the flexible conduit band the user interface device. The Busch device, however, has a number of drawbacks. For example, the two coupling members of the Busch magnetic coupler can only be disengaged from one another axially. As a result, the user is required to exert a substantial axial force to disengage the two-coupling members or the user must engage in a complicated rotational manipulation of the two coupling members relative to one another. As a result, Busch failed to improve on the conventional mechanical connections, particularly for users with impaired physical and/or mental ability. Further, neither Busch nor the mechanical prior art disclose a suitable means for storing the headgear and air hose when the gas flow system is not in use.

### SUMMARY

The present patent disclosure provides a gas flow delivery system for delivering a gas into headgear adapted to be worn by a user and magnetic coupling systems therefor that are designed to ameliorate one or more of the foregoing problems.

To this end, a gas flow delivery system for delivering a gas into headgear adapted to be worn by a user, is disclosed in which a magnetic coupling assembly is interposed in a fluid circuit between a gas source and an internal region of a headgear designed to receive at least the nose and/or mouth of a user.

In one embodiment, the gas delivery system comprises a gas source, headgear having a user interface configured to receive at least the nose and/or mouth of a user within an internal region thereof, and a fluid circuit in communication with the gas source at one end and the internal region of the user interface of the headgear at another end. Further, a magnetic coupling assembly is interposed in the fluid circuit. The magnetic coupling assembly includes a first magnetic coupling member and a second magnetic coupling member, where the first and second magnetic coupling members have opposing mating surfaces and define an axis along which the first and second magnetic coupling members magnetically engage one another.

Each of the first and second magnetic coupling members comprise a main body portion and a retaining cap connected to the main body portion, the retaining cap and main body portion of each coupling member define an annular chamber in which a ring-shaped magnet is disposed. The retaining caps of each of the first and second magnetic coupling members also define the opposing mating surfaces of the first and second coupling members. Preferably, at least a portion of the opposing mating surfaces are transverse to the axis.

In some embodiments, the headgear may comprise an interface input tube and the first magnetic coupling member is connected at one end to the interface input tube. The second magnetic coupling member may be connected at one end to a distal end of a gas delivery tube that defines at least part of the fluid circuit.

In some embodiments, the first magnetic coupling member is removably connectable to the interface input tube, for example, by way of an interference fit between one end of the first magnetic coupling member and an outer surface of the interface input tube. In other embodiments, however, the first magnetic coupling member may be integrally formed with the interface input tube or permanently connected thereto.

The second magnetic coupling member may be separated from the first magnetic coupling member through the application of an axial pull force. In some embodiments, the axial pull force must be an axial pull force of least 48 ounce-force or greater. If the first magnetic coupling member is removably connected to the interface input tube, the axial pull force required to separate the first and second magnetic coupling members is preferably configured to be less than the axial pull force required to separate the first coupling member from the interface input tube.

Preferably, the second magnetic coupling member may also be separated from the first magnetic coupling member through a torque created by the application of a force to the second coupling member orthogonal to the axis.

In some embodiments, the gas source may comprise an air fan.

In some embodiments, the second magnetic coupling member may include a connector configured to connect to a distal end of the gas delivery tube to the second coupling member. As an example, the connector included in the second magnetic coupling member may comprise threads designed to operatively engage with the external surface of a coil included in the gas delivery tube such that the second magnetic coupling member may be threaded onto the distal end of the distal end of the gas delivery tube.

Preferably the retaining cap and main body portion of each coupling member are made of plastic. The retaining cap and main body portion of each coupling member may be connected together by a variety of techniques, including, for example, sonic welding, mating threads, mating tongue and groove features, and/or an adhesive.

In some embodiments, the magnetic coupling assembly further comprises a docking station made from a ferromagnetic material. The docking station may be configured in size to magnetically hold the mating surface of at least one of the first and second coupling members against the docking station. Preferably, the docking station includes a docking side and a mounting side. The docking side preferably includes a flat area for receiving the mating surface of at least one of the first and second coupling members against the docking station. The mounting side preferably includes a double side adhesive or other suitable mounting means for mounting the docking station on, for example, a bedside table or other suitable surface.

In some embodiments, the magnetic coupling system may include two docking stations as described above, one for holding each of the first and second coupling members with the gas flow delivery system is not in use. In other embodiments, the docking station includes a first and a second docking area that are orthogonal to one another, each of the first and second docking areas being configured to magnetically hold the mating surface of at least one of the first and second coupling members against the docking station.

Further aspects, objects, desirable features, and advantages of the various inventions that are the subject of the present disclosure will become manifest and be better understood from the following description considered in connection with accompanying drawings in which various embodiments of the disclosed inventions are illustrated by way of example. It is to be expressly understood, however, that the drawings are for the purpose of illustration only and are not intended as a definition of the limits of any of the disclosed inventions.

### BRIEF DESCRIPTION OF THE DRAWINGS

FIG. 1 illustrates a positive gas flow delivery system used to deliver a flow of gas to an airway of a subject with a magnetic coupling assembly interposed in the gas flow delivery circuit.

FIG. 2 illustrates another positive gas flow delivery system used to deliver a flow of gas to an airway of a subject with a magnetic coupling assembly interposed in the gas flow delivery circuit. In the illustration of FIG. 2, the patient-side coupling member and the hose-side coupling member of the magnetic coupling assembly are decoupled from one another and magnetically connected to an optional docking station of the magnetic coupling assembly for storage during non-use and later retrieval by the patient.

FIG. 3 is a perspective view of a magnetic coupling according to one embodiment of the present patent disclosure.

FIG. 4 is a front view of the magnetic coupling of FIG. 3.

FIG. 5 is a rear view of the magnetic coupling of FIG. 3.

FIG. 6 is a right-side view of the magnetic coupling of FIG. 3.

FIG. 7 is a left-side view of the magnetic coupling of FIG. 3.

FIG. 8 is a partially exploded view of the magnetic coupling of FIG. 3 showing the patient-side coupling member and the hose-side coupling member decoupled from one another.

FIG. 9 is a cross-sectional view of the magnetic coupling assembly of FIG. 3 taken along cutline 9-9 in FIG. 4.

FIG. 10 is a cross-sectional view of the magnetic coupling assembly according to another embodiment.

FIG. 11 is a front view of the main body portion of the user-side coupling member of the magnetic coupling assembly of FIG. 10.

FIG. 12 is a perspective view of the retaining cap of the user-side coupling member of the magnetic coupling of FIG. 10. The retaining cap includes an annular shaped magnet disposed therein.

### DETAILED DESCRIPTION

While it should be understood that the inventions described herein are described in connection with particular examples, the scope of the inventions are not limited to the specific examples. Rather, those skilled in the art will appreciate after reviewing the present disclosure that the following teachings can be used in a much wider variety of applications than the examples specifically mentioned herein.

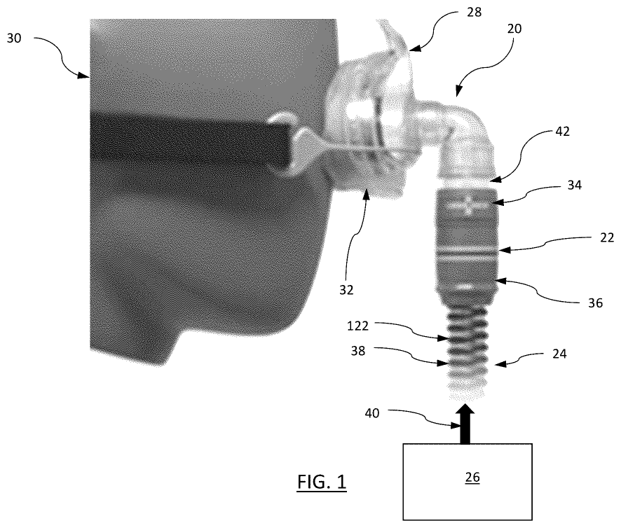

Referring now to the drawings in which like reference numerals designate like or corresponding components throughout the drawings, there is shown in FIG. 1 a gas flow delivery system 20 according to the present disclosure. The gas flow delivery system 20 is for delivering a gas into headgear 28 adapted to be worn by a user 30. The gas flow delivery system 20 includes a magnetic coupling assembly 22 interposed in a fluid circuit 24 between a gas source 26 and an internal region of a headgear 28 designed to receive at least the nose and/or mouth of a user 30.

A gas delivery tube 38 defines at least part of the fluid circuit 24 and is connected to the gas source 26 as illustrated by arrow 40.

According to the present embodiment, the gas delivery system 20 comprises a gas source 26, headgear 28 having a user interface 32 configured to receive at least the nose and/or mouth of a user within an internal region thereof, and a fluid circuit 24 in communication with the gas source at one end and the internal region of the user interface 32 of the headgear 28 at another end. Further, magnetic coupling assembly 22 is interposed in the fluid circuit 24. The magnetic coupling assembly 22 includes a first magnetic coupling member 34 and a second magnetic coupling member 36.

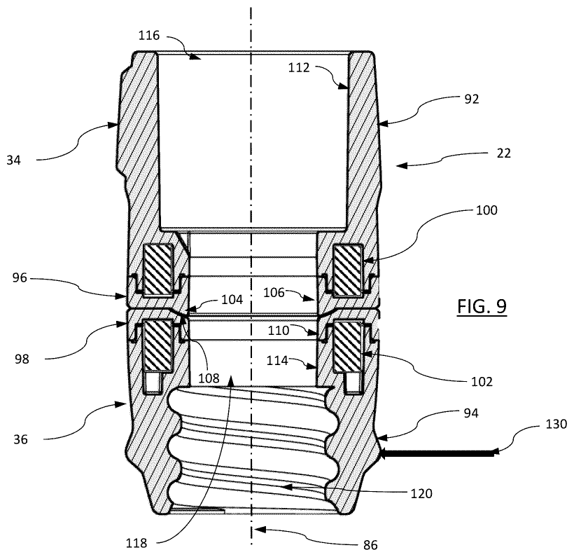

As best seen in FIGS. 8 and 9, the first and second magnetic coupling members 34, 36 have opposing mating surfaces 82, 84 and define an axis 86 along which the first and second magnetic coupling members magnetically engage one another.

The gas source 26 may comprise an air fan. The gas source 26 may also be any conventional ventilator, pressure support system, or other device that is used to communicate a flow of gas or gas at an elevated pressure above the ambient pressure to the airway of the user. Examples of such systems include, but are not limited to: a ventilator, continuous positive airway pressure (CPAP) device, or a variable pressure device, e.g. an auto-titrating device, proportional assist ventilation (PAV®) device, proportional positive airway pressure (PPAP®) device, C-Flex™device, Bi-Flex™ device, or a BiPAP® device, Other devices that communicate a flow of gas with an airway of a patient suitable for use in with the present invention include devices that apply a high and low or positive and negative pressure to the airway for purposes of secretion clearance or loosening.

The first magnetic coupling member 34 comprises a main body portion 92 and a retaining cap 96 connected to the main body portion 92. The retaining cap 96 and main body portion 92 of the first coupling member 34 defines an annular chamber in which a ring-shaped magnet 100 is disposed.

Similarly, the second magnetic coupling member 36 comprises a main body portion 94 and a retaining cap 98 connected to the main body portion 94. The retaining cap 98 and main body portion 94 of the second coupling member 36 defines an annular chamber in which a ring-shaped magnet 102 is disposed.

Magnets 100, 102 are arranged in their respective coupling members so that opposite North and South poles of the magnets 100, 102 are facing one another, and thus magnets 100, 102 will be attracted to one another when in the vicinity of each other. Preferably magnets 100, 102 are permanent magnets, more preferably rare earth magnets, such as neodymium (NdFeB) magnets.

The retaining caps 96, 98 of each of the first and second magnetic coupling members 34, 36, respectively, also define the opposing mating surfaces 82, 84 of the first and second coupling members 34, 36. Preferably, at least a portion of the opposing mating surfaces are transverse to the axis 86. As best seen in FIGS. 8 and 9, in the present embodiment, the majority of mating surfaces 82, 84 are orthogonal to axis 86. However, mating surface 82 includes a relatively small, tapered projection 104 located adjacent the inner diameter 106 of the retaining cap 96. Similarly, the mating surface 84 includes a matching tapered recess 108 adjacent the inner diameter 110 of the retaining cap 98 for receiving tapered projection 104.

Tapered projection 104 and corresponding tapered recess 108 cooperate to facilitate the automatic alignment of coupling member 34 relative to coupling member 36. While magnets 100, 102 will inherently bring coupling members 34, 36 together in proper alignment. Tapered projection 104 and tapered recess 108 will further facilitate this process as well as help prevent the inadvertent sideways displacement of second coupling member 36 relative to first coupling member 34 during use.

The inner diameter 106 of the retaining cap 96 and inner surface 112 of the main body portion 92 define a central bore 116 that extends through the first coupling member 34. Similarly, the inner diameter 110 of the retaining cap 98 and the inner surface 114 of the main body portion 94 define a central bore 118 that extends through the second coupling member 36.

Central bores 116 and 118 provide part of the fluid circuit 24 for communicating gas flow from gas source 26 to the internal region of user interface 32 of headgear 28.

To complete the fluid circuit 24, user interface 32 of headgear 38 comprises an interface input tube 42 in the present embodiment. The first magnetic coupling member 34 is connected at one end to the interface input tube 42. The second magnetic coupling member 36 is connected at one end to a distal end of a gas delivery tube 38 that defines at least part of the fluid circuit 24.

Preferably, the first magnetic coupling member 34 is removably connectable to the interface input tube 42 to facilitate cleaning. For example, the first magnetic coupling member 34 may be removably connected to the interface input tube 42 by way of an interference fit between the internal surface 112 of first magnetic coupling member 34 at an end opposite mating end 82 and an outer surface of the interface input tube 42.

While a removable interference fit is employed in the present embodiment, other removable connections are also contemplated. Further, in other embodiments, the first magnetic coupling member 34 may be integrally formed with the interface input tube 42 or permanently connected thereto.

The second magnetic coupling member 34 may include a connector configured to connect to the distal end of the gas delivery tube 38. As an example, the connector included in the second magnetic coupling member 36 may comprise threads 120 designed to operatively engage with the external surface of a coil 122 included in the gas delivery tube 38 (see FIGS. 1 and 9) such that the second magnetic coupling member 36 may be threaded onto the distal end of the gas delivery tube 38.

Preferably the retaining caps 96, 98 and main body portions 92, 94 of each coupling member 34, 36, respectively, are made of plastic. This will permit the retaining caps 96, 98 and main body portions 92, 94 to be readily injection molded or 3D printed. The plastic material selected may also be selected so that the retaining caps 96, 98 of each of the coupling members 34, 34 may be connected to their corresponding main body portion 92, 94, respectively, via sonic welding. Other connection techniques may also be employed, including, for example, the use of mating threads, mating tongue and groove locking features, and/or an adhesive.

The second magnetic coupling member 36 may be separated from the first magnetic coupling member 34 through the application of an axial pull force. In some embodiments, the axial pull force must be an axial pull force of least 48 ounce-force or greater. If the first magnetic coupling member is removably connected to the interface input tube 42, the axial pull force required to separate the first and second magnetic coupling members 34, 36 is preferably configured to be less than the axial pull force required to separate the first coupling member 34 from the interface input tube 42.

Significantly, the design of the magnetic coupling assembly 22 according the present embodiment also permits the second magnetic coupling member 36 to be separated from the first magnetic coupling member 34 through a torque created by the application of a force to the second coupling member 36 orthogonal to the axis 86, such as force in the direction of arrow 130 in FIG. 9. Tapered projection 104 and corresponding tapered recess 108 are configured so not to interfere with decoupling of second coupling member 36 from first coupling member 34 through a torque created by a force applied orthogonal to axis 86.

Further, the first and second coupling members 34, 36 of the magnetic coupling assembly 22 may be configured to permit user 30 to couple and uncouple the coupling members 34, 36 with a single hand and without actually viewing the coupling members 34, 36 when they are to be coupled together or uncoupled. For example, the first and second coupling members 34, 36, may be configured so that magnetic force of attraction between the two coupling members 34, 36 is such that the user 30 need only bring the two coupling members into proximity with (although not necessarily even touching) one another and the magnetic force of attraction between the two coupling members 34, 36 will automatically align and couple the members 34, 36 together. As a result, user 30 need not be able to visualize the first and second coupling members 34, 36 of magnetic coupling assembly 22 when coupling or uncoupling them. Furthermore, as the strength of the magnetic force of attraction between the coupling members 34, 36 is increased, then user 30 will not need to bring the coupling members 34, 36 as close together in order for the magnetic force of attraction between the two coupling members 34, 36 to automatically align and couple the members 34, 36 together. Further, as previously noted, the tapered projection 104 and corresponding recess 108 will also help with the alignment of the coupling members 34, 36 when coupling them together.

The user 30 can also rely on the haptic feedback provided by the magnetic force of attraction between the two coupling members 34, 36 to know when he or she has brought the second coupling member 36 sufficiently close to, and sufficiently aligned with, first coupling member 34 so as to release the second coupling member 36 and allow the magnetic force of attraction between the two coupling members 34, 36 to finish aligning and coupling the members 34, 36 together in a fluid-tight manner.

The strength of the magnetic force of attraction between the two coupling members 34, 36 can also be set so that when the two coupling members 34, 36 couple together as a result of the magnetic force of attraction that a distinct, audible noise, such as an audible “clacking” noise, will be made due to the two coupling members coming together in a fluid-tight manner. As a result, user 30 can listen for the clacking or other distinct noise to verify that coupling members 34, 36 have been properly coupled together in a fluid-tight manner without ever visualizing the two coupling members when coupling them together.

Furthermore, the inclusion of a magnetic coupling assembly 22 in the fluid circuit 24 also substantially increases the safety of the gas flow delivery system 20 over conventionally known systems. For example, while the first and second coupling members 34, 36 may be configured so that magnetic force of attraction between the two coupling members 34, 36 is sufficient to automatically align and couple the members 34, 36 together when they are brought into proximity to one another, the force of attraction may also be set so that the amount of force required to disconnect the male and female coupling members 34, 36 is such that the coupling members will disconnect without injuring the user 30 in the event that a portion of the tubing 38 is snagged on an object or otherwise subjected to a force while the user 30 is wearing headgear 28.

The magnetic force of attraction between coupling members 34, 36 may be increased, for example, by (i) increasing the thickness of magnets 100, 102; (ii) increasing the cross-sectional area of the pole of magnets 100, 102 that faces the other magnetic material (“the mating cross-sectional area”); (iii) increasing the flux density (B) and/or magnetization (131) of the magnetic material used to make the magnets 100, 102; and/or (iv) decreasing the thickness and/or magnetic permeability (μ) of any non-magnetic material between the magnets 100, 102 and the mating surfaces 82, 84 of first and second coupling members 34, 36, respectively. Conversely, the magnetic force of attraction between coupling members 34, 36 may be decreased, for example, by adjusting parameters (i)-(iv) in the opposite direction.

In some approaches, an axial pull force that is greater than 48 ounce-force and less than 160 ounce-force between the first coupling member 34 and second coupling member 36 is required to decouple the male and female coupling members. In other approaches, an axial pull force that is greater than 96 ounce-force and less than 128 ounce-force between the first coupling member 34 and second coupling member 36 is required to decouple the first and second coupling members. In still other approaches, an axial pull force that is greater than 100 ounce-force and less than 120 ounce-force between the first coupling member 34 and second coupling member 36 is required to decouple the first and second coupling members.

Importantly, the magnitude of the force applied orthogonally (e.g., force 130) to decouple the first and second coupling members via a torque will be less than the required axial pull force required to separate the two coupling members, particularly the longer the moment arm between the point of application of the force 130 and mating surfaces 84, 86. This is because the further the force 130 is applied away from mating surfaces 84, 86, the greater the moment arm and hence the less force required to create the necessary torque to uncouple the first and second coupling members 34, 26.

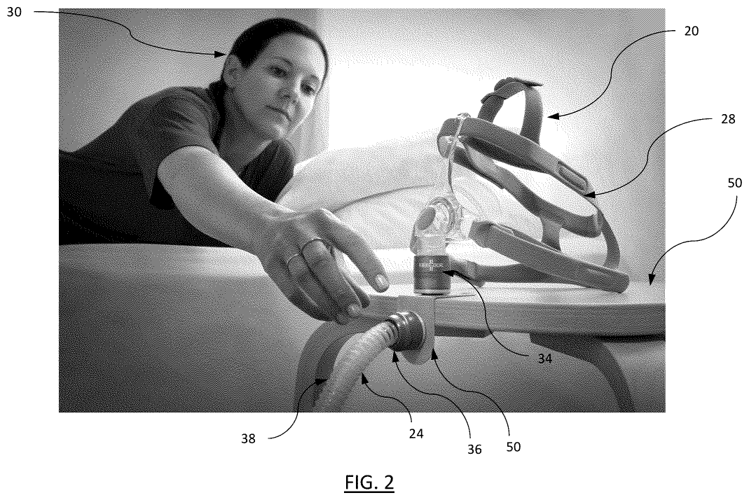

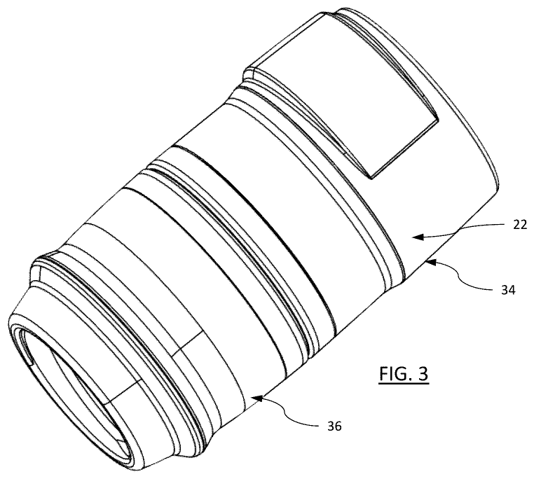

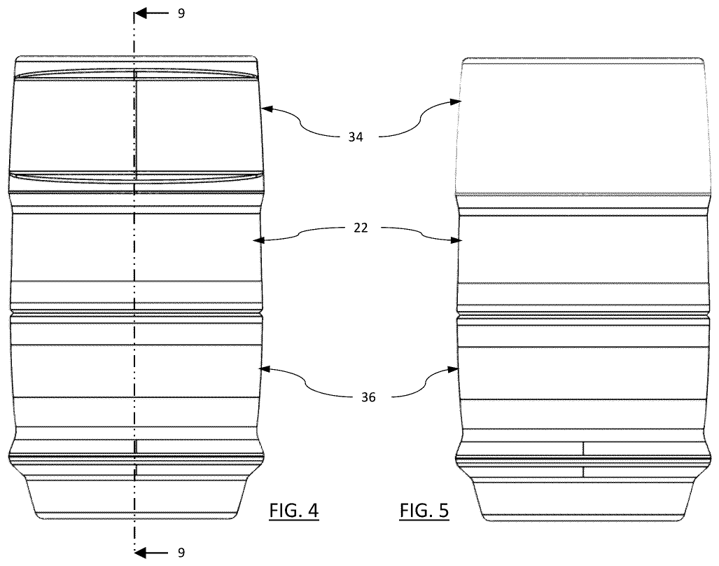

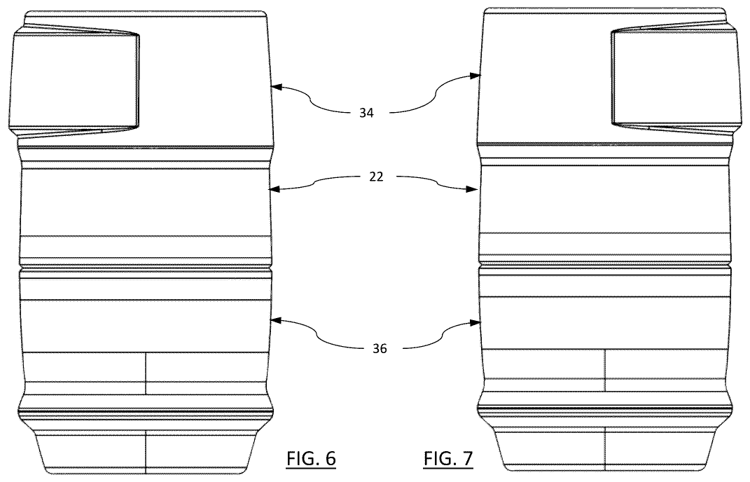

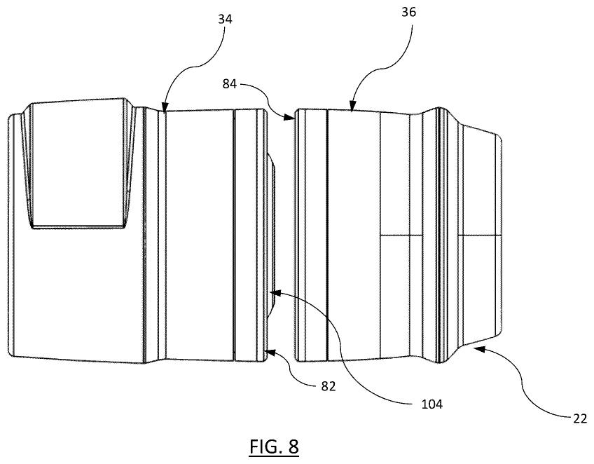

As shown in FIG. 2, in some embodiments, the magnetic coupling assembly 22 further comprises a docking station 50 made from a ferromagnetic material. The docking station 50 may be configured in size to magnetically hold the mating surface 82, 84 of at least one of the first and second coupling members 34, 36 against the docking station 50. Preferably, the docking station 50 includes a docking side and a mounting side. The docking side preferably includes a flat area for receiving the mating surface 82, 84 of at least one of the first and second coupling members 34, 26 against the docking station. The mounting side preferably includes a double side adhesive or other suitable mounting means for mounting the docking station 50 on the upper surface of, for example, a bedside table 52 or other suitable surface.

In the present embodiment, the docking station 50 includes a first and a second docking area that are orthogonal to one another, each of the first and second docking areas are configured to magnetically hold the mating surface 82, 84 of at least one of the first and second coupling members 34, 36 against the docking station. This allows user 30 to easily store the first magnetic coupling member 34 along with its attached headgear 28 and the second magnetic coupling member 36 with its attached gas hose 38 when the gas flow delivery system 20 is not in use.

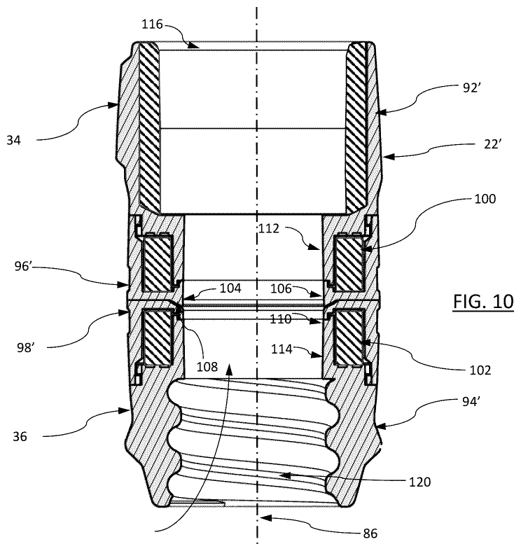

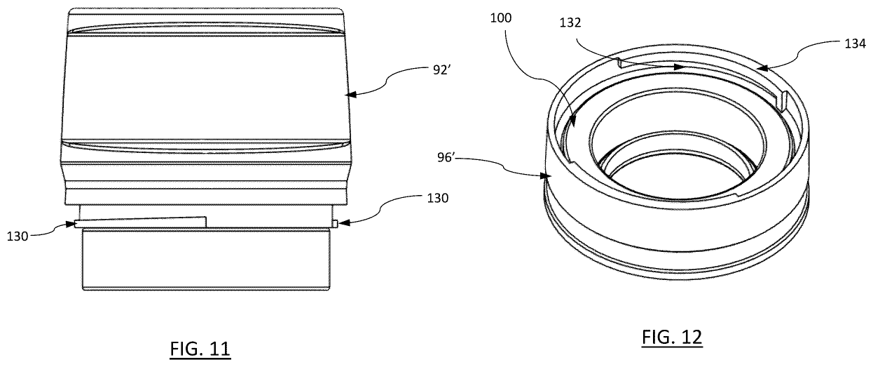

An alternative embodiment of a magnetic coupling assembly 22′ according to the present disclosure is shown in FIGS. 10-12. The coupling assembly 22′ shown in FIGS. 10-12 shares many of the same features as magnetic coupling assembly 22 previously described. One main difference is that the main body portion 92′ and retaining cap 96′ of the first coupling member 34 are attached to one another using a tongue and groove style connector. This can be best seen in FIGS. 11 and 12 in which two inclined or tapered tongues 130 are positioned on opposite sides of a mating surface of the main body portion 92′. Conversely, the retaining cap 96′ includes mating tapered grooves 132 formed by bosses 134 provided on opposite sides of the inner mating surface of the retaining cap 96′. To connect the retaining cap 96′ to the main body portion 92′ the tongues 130 are aligned with the gap between the bosses 134 in retaining cap 96′ and then rotated clockwise to enter the groove 132. Tongues 130 then rotatably engage with bosses 134 to removably lock the retaining cap onto the main body portion. An adhesive can be added to make a more permanent connection between the retaining cap 96′ and main body portion 92′.

The main body portion 94′ and retaining cap 98′ are connected to one another in the same manner as just described.

The first coupling member 34 of the present embodiment is also different in that it includes an optional silicon sleeve 116 for making the interference fit connection with the interface input tube 42.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed:

1. A gas flow delivery system for delivering a gas into headgear adapted to be worn by a user, the gas delivery system comprising:
a gas source;
headgear having a user interface configured to receive at least the nose and/or mouth of a user within an internal region thereof;
a fluid circuit in communication with the gas source at one end and the internal region of the user interface of the headgear at another end;
a magnetic coupling assembly interposed in the fluid circuit, the magnetic coupling assembly including a first magnetic coupling member and a second magnetic coupling member, the first and second magnetic coupling members having opposing mating surfaces and defining an axis along which the first and second magnetic coupling members magnetically engage one another, wherein:
each of the first and second magnetic coupling members comprise a main body portion and a retaining cap connected to the main body portion, the retaining cap and main body portion of each coupling member defining an annular chamber in which a ring-shaped magnet is disposed;
the retaining caps of each of the first and second magnetic coupling members define the opposing mating surfaces of the first and second coupling members, at least a portion of the opposing mating surfaces being transverse to the axis;
the headgear comprises an interface input tube and the first magnetic coupling member is connected to the interface input tube;
the second magnetic coupling member is connected to a gas delivery tube that defines at least part of the fluid circuit;
the second magnetic coupling member may be separated from the first magnetic coupling member through the application of an axial pull force of at least 48 ounce-force or greater; and
wherein the second magnetic coupling member may also be separated from the first magnetic coupling member through a torque created by the application of a force orthogonal to the axis to the second magnetic coupling member.
a detachment force required to disconnect the second magnetic coupling member from the first magnetic coupling member is such that the coupling members will disconnect without injuring a user wearing the headgear if a portion of the gas delivery tube connected to the second magnetic coupling member is subjected to a force exceeding such detachment force.

2. A gas flow delivery system according to claim 1, wherein the gas source comprises an air fan.

3. A gas flow delivery system according to claim 1, wherein the first magnetic coupling member is removably connectable to the interface input tube.

4. A gas flow delivery system according to claim 1, wherein the first magnetic coupling member is removably connectable to the interface input tube via an interference connection.

5. A gas flow delivery system according to claim 1, wherein the first magnetic coupling member is integrally formed with the interface input tube.

6. A gas flow delivery system according to claim 1, wherein the second magnetic coupling member includes a connector configured to connect to a distal end of the gas delivery tube to the second coupling member.

7. A gas flow delivery system according to claim 6, wherein the connector included in the second magnetic coupling member comprises threads designed to engage with the external surface of a coil included in the gas delivery tube.

8. A gas flow delivery system according to claim 1, wherein the retaining cap and main body portion of each coupling member are made of plastic.

9. A gas flow delivery system according to claim 8, wherein the retaining cap and main body portion of each coupling member are connected together by sonic welding.

10. A gas flow delivery system according to claim 8, wherein the retaining cap and main body portion of each coupling member are connected together by a sonic weld, mating threads, or a mating tongue and groove features.

11. A gas flow delivery system according to claim 1, wherein the magnetic coupling assembly further comprises a docking station made from a ferromagnetic material, the docking station configured to magnetically hold the mating surface of at least one of the first and second coupling members against the docking station.

12. A gas flow delivery system according to claim 11, wherein the docking station includes first and second docking area that are orthogonal to one another, each of the first and second docking areas being configured to magnetically hold the mating surface of at least one of the first and second coupling members against the docking station.
